(19) United States
(12) Patent Application Publication
(10) Pub. No.: US 2026/0014214 A1
(43) Pub. Date: Jan. 15, 2026

(54) PROBIOTIC COMPOSITIONS AND THERAPEUTIC USES THEREOF

(71) Applicant: MULTIGERM UK ENTERPRISES LTD, Surrey (GB)

(72) Inventors: Michael BUTLER, Surrey (GB); Samantha GILL, Surrey (GB)

(73) Assignee: MULTIGERM UK ENTERPRISES LTD, Surrey (GB)

(21) Appl. No.: 18/994,283

(22) PCT Filed: Jul. 18, 2023
(86) PCT No.: PCT/EP2023/069926

§ 371 Date: Jan. 14, 2025

(30) Foreign Application Priority Data
Jul. 18, 2022 (GB) 2210523.3

(51) Int. Cl. A61K 35/744; A61K 31/353; A61K 36/45; A61K 47/26; A61P 13/02

(52) U.S. Cl. CPC A61K 35/744; A61K 31/353; A61K 36/45; A61K 47/26; A61P 13/02

(57) ABSTRACT

The present invention relates to liquid, non-dairy probiotic compositions comprising a viable population of lactic acid bacteria, mannose and/or a proanthocyanidin, and medical uses thereof, particularly in treating/preventing urinary tract infection. The present invention also relates to kits comprising a liquid, non-dairy composition comprising a population of viable lactic acid bacteria and mannose.

### FIELD OF INVENTION

The invention relates to liquid, non-dairy probiotic compositions comprising a viable population of lactic acid bacteria, and medical uses thereof, particularly in treating/preventing urinary tract infection.

### BACKGROUND

Urinary tract infection (UTI) is infection usually caused by bacteria, but also includes infection by other microorganisms such as fungi, viruses, or parasites. UTI results from pathogenic organisms gaining access to the urinary tract without being eliminated effectively. Entry into the urinary tract may occur directly (e.g. via insertion of a catheter into the bladder, instrumentation, or surgery), via the blood stream (more likely in immunocompromised people), or retrograde (ascending through the urethra).

UTI may affect any part of the urinary tract, including the bladder (e.g. cystitis), urethra (e.g. urethritis), ureters and/or kidneys (e.g. ureteritis, pyelitis, pyelonephritis and nephritis). UTI may also affect the testes and other reproductive organs (e.g. epididymitis, orchitis, and prostatitis). Common symptoms of UTI include pain (e.g. a burning sensation) when urinating (dysuria), frequent urge to urinate, lower back pain and fever. Presence of bacteria in the urine may also be classified as bacteriuria, which may be symptomatic or asymptomatic.

The most common uropathogen causing UTI is Escherichia coli, accounting for approximately 70-95% of cases in adults and about 85% or more of cases in children (National Institute for Health and Care Excellence [NICE], 2022, Clinical Knowledge Summary: “Urinary tract infection (lower)-men”; NICE, 2021, Clinical Knowledge Summary: “Urinary tract infection (lower)-women”; NICE, 2019, Clinical Knowledge Summary: “Urinary tract infection-children”). Other causative micro-organisms include Staphylococcus saprophyticus, Klebsiella species, Proteus mirabilis, Enterobacter species, Enterococcus species, Serratia marcescens, Pseudomonas species, Staphylococcus aureus, and Candida albicans.

Acute UTI occurs in up to 50% of females, and estimates suggest that by the age of 24 years, nearly one third of females will have had at least one episode of cystitis (NICE, 2021, Clinical Knowledge Summary: “Urinary tract infection (lower)-women”). Notably, approximately 25-35% of women with UTI have a recurrent infection within 3 to 6 months, and approximately 44% of women have a recurrence within 12 months.

While UTI is less common in the male population, the incidence of UTI in males increases substantially with increasing age, co-existing illness, institutional care, and indwelling urinary catheter use. Particularly due to urinary catheter use, UTI is the most common hospital-acquired infection in the UK, accounting for 23% of all hospital-acquired infections (NICE, 2022, Clinical Knowledge Summary: “Urinary tract infection (lower)-men”).

Aside from typical UTI, it is possible for bacteria to colonise the urinary tract without traditional symptoms or signs of infection (e.g. asymptomatic bacteriuria). Asymptomatic bacteriuria is more common in elderly populations with comorbidities, in institutional care, and/or with an indwelling urinary catheter. The reported prevalence of asymptomatic bacteriuria in men older than 70 years of age ranges from approximately 4-7% (NICE, 2022, Clinical Knowledge Summary: “Urinary tract infection (lower)-men”). The prevalence of asymptomatic bacteriuria in institutionalized elderly populations ranges from 19-37%.

Antibiotics are usually the first line treatment for UTIs and asymptomatic bacteriuria. Patients with recurrent UTI may be prescribed antibiotic prophylaxis. However, this evidently presents a significant risk of antibiotic resistance. Indeed, a 2016 study found that of over 1 million UTI samples, 34% were found to be resistant to Trimethoprim which was once the first choice treatment for UTI in England (Public Health England, 2017, “English Surveillance Programme For Antimicrobial Utilisation And Resistance (ESPAUR) Report”; NICE, 2017, “Antibiotic resistance is now ‘common’ in urinary tract infections”). Similar trends in antimicrobial resistance have been reported in relation to other UTI pathologies, for example increased antifungal resistance in Candida species responsible for urinary tract candidiasis (Behzadi et al., 2015, “Urinary Tract Infections and Candida Albicans”, Cent European J Urol., vol. 68, no. 1, p. 96-101).

Considering the high rates of recurrent UTI and the increasing emergence of antimicrobial-resistant uropathogens, there is a need for new UTI therapies.

### SUMMARY OF INVENTION

In a first aspect, the invention provides a liquid, non-dairy probiotic composition comprising a population of viable lactic acid bacteria, and mannose.

In a second aspect, the invention provides a liquid, non-dairy probiotic composition comprising a population of viable lactic acid bacteria, and a proanthocyanidin (PAC).

In a third aspect, the invention provides a liquid, non-dairy probiotic composition comprising a population of viable lactic acid bacteria, a proanthocyanidin (PAC), and mannose.

In certain embodiments of the first, second and third aspects, the population of viable lactic acid bacteria comprises one or more Lactobacillus species. In preferred embodiments, the population of viable lactic acid bacteria comprises one or more of Lactobacillus rhamnosus, Lactobacillus acidophilus, and Lactobacillus plantarum bacteria.

In certain embodiments, the population of viable lactic acid bacteria comprises one or more Enterococcus species. In preferred embodiments, the population of viable lactic acid bacteria comprises Enterococcus faecium bacteria.

In certain preferred embodiments, the population of lactic acid bacteria comprises Lactobacillus rhamnosus, Lactobacillus acidophilus, and Lactobacillus plantarum bacteria. Preferably, the population of lactic acid bacteria comprises Lactobacillus rhamnosus, Lactobacillus acidophilus, Lactobacillus plantarum and Enterococcus faecium bacteria.

In preferred embodiments, the lactic acid bacteria are metabolically active.

In certain embodiments of the first and third aspects, the concentration of mannose in the probiotic composition is about 2.8 mg/mL to about 280 mg/mL. In certain such embodiments, the concentration of mannose is about 5.6 mg/ml to about 140 mg/mL. In certain such embodiments, the concentration of mannose is about 11 mg/mL to about 70 mg/mL. In preferred embodiments, the concentration of mannose in the probiotic composition is about 28 mg/mL.

In certain embodiments of the second and third aspects, the PAC concentration in the probiotic composition is about 0.04 mg/mL to about 5 mg/mL. In certain such embodiments, the PAC concentration is about 0.1 mg/mL to about 2.5 mg/mL. In certain such embodiments, the PAC concentration is about 0.2 mg/mL to about 1.3 mg/mL. In certain such embodiments, the PAC concentration is about 0.4 mg/mL to about 0.6 mg/mL. In preferred embodiments, the PAC concentration in the probiotic composition is about 0.5 mg/mL.

In certain embodiments, the PAC is obtainable from cranberries.

In certain embodiments, the probiotic composition comprises a cranberry product. In preferred such embodiments, the cranberry product comprises a PAC. In certain embodiments, the cranberry product is a cranberry extract. A cranberry extract may comprise a cranberry juice (i.e. a soluble fraction), a cranberry pomace (i.e. an insoluble fraction), or both a cranberry juice and a cranberry pomace. In preferred embodiments, the cranberry extract comprises or consists of cranberry juice. In certain embodiments, the cranberry extract comprises cranberry juice and one or more excipients.

In preferred embodiments, the cranberry product (e.g. a cranberry extract, for example a cranberry juice) is present in the composition in an amount sufficient to provide the probiotic composition with a PAC concentration of about 0.04 mg/mL to about 5 mg/mL, preferably about 0.1 mg/mL to about 2.5 mg/mL, more preferably about 0.2 mg/mL to about 1.3 mg/mL, even more preferably about 0.4 mg/mL to about 0.6 mg/mL, most preferably about 0.5 mg/mL.

In certain embodiments, the cranberry product (e.g. a cranberry extract, for example a cranberry juice) is present in the probiotic composition at a concentration of about 0.1 mg/mL to about 14 mg/mL. In certain such embodiments, the cranberry product is present at a concentration of about 0.3 mg/mL to about 7 mg/mL. In certain such embodiments, the cranberry product is present at a concentration of about 0.6 mg/ml to about 3.5 mg/mL. In certain such embodiments, the cranberry product is present at a concentration of about 1 mg/mL to about 1.8 mg/mL. In preferred embodiments, the cranberry product is present in the probiotic composition at a concentration of about 1.4 mg/mL.

In certain embodiments of the first, second and third aspects, the probiotic composition comprises a disaccharide, oligosaccharide or polysaccharide comprising at least one mannose residue.

In certain embodiments, the probiotic composition comprises a disaccharide, oligosaccharide or polysaccharide comprising at least one mannose residue, wherein the mannose concentration thereof is about 2.8 mg/mL to about 280 mg/mL. In certain such embodiments, the mannose concentration is about 5.6 mg/mL to about 140 mg/mL. In certain such embodiments, the mannose concentration is about 11 mg/mL to about 70 mg/mL. In preferred such embodiments, the mannose concentration is about 28 mg/mL. The “mannose concentration thereof” refers to the total concentration of mannose in the probiotic composition.

In a fourth aspect, the invention provides a liquid, non-dairy probiotic composition comprising a viable population of lactic acid bacteria for use in a method of treatment or prevention of urogenital infection and/or urogenital inflammation.

In a fifth aspect, the invention provides a liquid, non-dairy probiotic composition according to the first, second or third aspects of the invention, which is for use in a method of treatment or prevention of urogenital infection and/or urogenital inflammation.

In preferred embodiments of the fourth and fifth aspects of the invention, the probiotic composition is for use in treating or preventing a urogenital infection. In most preferred such embodiments, the composition is for use in treating or preventing a urinary tract infection.

In a sixth aspect, the invention provides a method of treating or preventing urogenital infection and/or urogenital inflammation in a subject in need thereof, the method comprising administering a liquid, non-dairy probiotic composition comprising a viable population of lactic acid bacteria to the subject.

In a seventh aspect, the invention provides a method of treating or preventing urogenital infection and/or urogenital inflammation in a subject in need thereof, the method comprising administering to the subject a probiotic composition as defined in respect of any of the first, second or third aspects of the invention.

In preferred embodiments of the sixth and seventh aspects, the method is a method of treating or preventing a urogenital infection. In most preferred such embodiments, the method is a method of treating or preventing a urinary tract infection.

Unless specified otherwise herein, all embodiments of the fourth and fifth aspects are equally applicable to the sixth and seventh aspects of the invention and vice-versa.

In an eighth aspect, the invention provides a kit comprising:
(i) a first liquid, non-dairy composition comprising a population of viable lactic acid bacteria; and
(ii) a second composition comprising mannose.

In certain embodiments, the kit comprises a third composition comprising a PAC.

In certain embodiments, any two of the first, second and third compositions of the kit are formulated in combination (i.e. co-formulated). In certain embodiments, the first composition is formulated in combination with the second composition. In other embodiments, the first composition is formulated in combination with the third composition. In other embodiments, the second composition is formulated in combination with the third composition.

In certain embodiments of the kit, the first, second and third compositions are co-formulated in a single composition. In alternative embodiments, the first, second and third compositions are each formulated separately.

The invention can also be understood by reference to the following numbered clauses.
1. A liquid, non-dairy probiotic composition comprising a population of viable lactic acid bacteria, and a proanthocyanidin (PAC).
2. The composition of clause 1, wherein the population of viable lactic acid bacteria comprises one or more Lactobacillus species.
3. The composition of clause 1 or clause 2, wherein the population of viable lactic acid bacteria comprises one or more of Lactobacillus rhamnosus, Lactobacillus acidophilus, and Lactobacillus plantarum bacteria.
4. The composition of any preceding clause, wherein the population of viable lactic acid bacteria comprises one or more Enterococcus species.
5. The composition of any preceding clause, wherein the population of viable lactic acid bacteria comprises Enterococcus faecium bacteria.
6. The composition of any preceding clause, wherein the population of viable lactic acid bacteria comprises each of Lactobacillus rhamnosus, Lactobacillus acidophilus, Lactobacillus plantarum and Enterococcus faecium bacteria.
7. The composition of any preceding clause, wherein the PAC is obtainable from cranberries.
8. The composition of any preceding clause, comprising a cranberry product comprising said PAC, preferably wherein the cranberry product is a cranberry extract.
9. The composition of any preceding clause comprising mannose.
10. The composition of any preceding clause comprising a disaccharide, oligosaccharide or polysaccharide comprising at least one mannose residue.
11. The composition of any preceding clause, wherein the lactic acid bacteria are metabolically active.
12. The composition of any preceding clause, wherein the PAC is present at a concentration of about 0.04 mg/mL to about 5 mg/mL, optionally about 0.1 mg/mL to about 2.5 mg/mL, optionally about 0.2 mg/mL to about 1.3 mg/mL, optionally about 0.4 mg/mL to about 0.6 mg/mL, preferably about 0.5 mg/mL.
13. The composition of any preceding clause, wherein the PAC is comprised in a cranberry extract, wherein the cranberry extract is present at a concentration of about 0.1 mg/mL to about 14 mg/mL, optionally about 0.3 mg/mL to about 7 mg/mL, optionally about 0.6 mg/mL to about 3.5 mg/mL, optionally about 1 mg/mL to about 1.8 mg/mL, preferably about 1.4 mg/mL.
14. The composition of any preceding clause, comprising mannose or a disaccharide, oligosaccharide or polysaccharide comprising a mannose residue, wherein the mannose concentration thereof is about 2.8 mg/mL to about 280 mg/mL, optionally about 5.6 mg/mL to about 140 mg/mL, optionally about 11 mg/mL to about 70 mg/mL, preferably about 28 mg/mL.
15. A liquid, non-dairy probiotic composition comprising a viable population of lactic acid bacteria for use in a method of treatment of urogenital infection and/or urogenital inflammation.
16. The composition for use according to clause 15, wherein the composition is for use in treating a urogenital infection, wherein said urogenital infection is a urinary tract infection.
17. The composition for use according to clause 15 or clause 16, wherein the composition is as defined in any of clauses 1-14.
18. A kit comprising:
   (i) a first liquid, non-dairy composition comprising a population of viable lactic acid bacteria;
   (ii) a second composition comprising a PAC; and/or
   (iii) a third composition comprising mannose, or a disaccharide, oligosaccharide or polysaccharide comprising at least one mannose residue.
19. The kit of clause 18, wherein the population of lactic acid bacteria is as defined in any one of clauses 2-6.
20. The kit of clause 18 or clause 19, wherein any two or all three of the first, second and third compositions are formulated in combination.

### BRIEF DESCRIPTION OF FIGURES

FIG. 1: Kaplan-Meier curves showing the time to first reported UTI symptoms for the intervention (“A”; n=65) and control (“B”; n=74) groups.

### DETAILED DESCRIPTION

### Definitions

The terms as used herein are given their conventional definition in the art as understood by the skilled person, unless otherwise defined below. In the case of any inconsistency or doubt, the definition as provided herein should take precedence.

Any reference referred to as being “incorporated herein” is to be understood as being incorporated in its entirety.

As used in this specification, the singular forms “a,” “an,” and “the” include plural referents unless expressly and unequivocally limited to one referent. The term “or” is used interchangeably with the term “and/or” unless the context clearly indicates otherwise.

The term “lactic acid bacteria (LAB)” refers to a group of Gram positive, catalase negative, non-motile anaerobic bacteria that ferment carbohydrates to lactic acid. This group includes the genera Lactobacillus, Lactococcus, Pediococcus, Bifidobacterium, and Enterococcus. Exemplary probiotic lactic acid bacteria include, but are not limited to, those in the genera Lactobacillus and Enterococcus.

“Proanthocyanidins”, interchangeably referred to herein as “PACs”, are a class of flavonoid, specifically oligomers (2-5 units) or polymers (more than 5 units) of monomeric flavan-3-ols.

As used herein, a “PAC” preferably refers to an A-type proanthocyanidin and/or a B-type proanthocyanidin, most preferably an A-type proanthocyanidin. A-type and B-type PACs can be differentiated by their interflavanic linkages. A-type PACs comprise at least one double interflavanic linkage consisting of a C-C bond and an ether (R′—O-R″) bond. A-type and B-type PAC linkages are further described in the art (see, e.g., I. Ky, et al., Tannins, Editor(s): Benjamin Caballero, Paul M. Finglas, Fidel Toldrá, Encyclopedia of Food and Health, Academic Press, 2016, p. 247-255). Preferably, the PACs referred to herein are water-soluble. Soluble PAC levels can be measured via routine methods.

A PAC may be obtained from a number of commercial sources, including plant-based food sources. For example, PACs may be obtained from cranberry, lingonberry, black elderberry, black chokeberry, black currant, blueberry, bilberry, persimmon, banana, carob bean, Chinese quince, rose hip, medlar, mulberry, plum, apricot, walnut, hazelnut, peanut, silverberry, apple, pear, pine bark, aronia fruit, cocoa bean, açaí, tea leaves, and pomegranate. Methods of isolating, identifying and characterising PACs are well known, including for example standard chromatographic, colorimetric and spectrometric techniques.

A PAC may be added to the composition either directly in a purified form, or comprised in another product. For example, the PAC may be comprised in a plant product.

A “plant product” (e.g. a cranberry product) as used herein is preferably an edible plant product which is, or is derived from, the fruit, seed or leaf of a plant, and which is in a form suitable for formulation in the probiotic composition of the invention. For example, the plant product may be a plant extract (e.g. a cranberry extract). In another example, the plant product may be an entire fruit (e.g. a freeze-dried/powdered cranberry).

A “plant extract” (e.g. a cranberry extract) as used herein preferably refers to a substance extracted from a raw plant material (e.g. a plant product), for example by pressing, crushing, juicing, steeping, decoction, maceration, distillation and/or other separation process. A plant extract (e.g. a cranberry extract) may comprise a juice (i.e. a soluble fraction) and/or a pomace (i.e. an insoluble fraction). The juice fraction is typically extracted from the fruit of the plant. The pomace fraction is typically composed of fruit skins, seeds and/or stems.

A “cranberry extract” may comprise a cranberry juice, a cranberry pomace, or both a cranberry juice and a cranberry pomace.

“Mannose” as used herein preferably relates to the D-mannose enantiomer.

“Oligosaccharides” are saccharide polymers containing 3 to 10 saccharide units; whereas the term “polysaccharides” includes longer polymeric structures, such as those formed from repeating saccharide (or disaccharide) units.

The term “simple sugars” as used herein refers to both monosaccharides and disaccharides, unless otherwise stated.

The term “reducing sugars” as used herein refers to sugars which either have an aldehyde group or are capable of forming an aldehyde group in solution through isomerisation. The presence of reducing sugars may be determined by means of the Nelson-Somogyi method using glucose as the reference standard (Somogyi, M. (1952) Journal of Biological Chemistry., vol. 195., p. 19; reproduced in many standard textbooks of carbohydrate chemistry). Although certain complex carbohydrates (e.g. starches) may contain reducing ends, and therefore fulfil the definition of “reducing sugars”, a determination of the content of “reducing sugars” in a given sample (e.g. a sample of probiotic composition as described herein) using the Nelson-Somogyi method may be taken as an approximation of the amount of simple sugars in the sample, since the simple sugars contain a greater proportion of reducing ends per unit mass than complex carbohydrates.

The terms “total carbohydrate” or “total carbohydrate content” as used herein refer to the total amount of complex carbohydrate and simple sugars present in a given product (e.g. a probiotic composition as described herein). Total carbohydrate content may be measured using the phenol-sulphuric acid assay, using glucose as a reference standard (Dubois, M., Gilles, K. A., Hamilton, J. K., Rebers, P. A. and Smith, F. (1956) Analytical Chemistry, vol. 28., p. 350).

The term “non-dairy” as used herein refers to products which do not contain and are not based upon milk from a mammal, in accordance with the definition accepted in the art. Accordingly, non-dairy products do not contain and are not based upon milk, butter, cheese (including vegetarian cheese), yoghurt, cream, milk powder, whey, lactose, lactoproteins (including caseins and caseinates), anhydrous milk fat or kefir.

A “subject” as used herein refers to a mammalian, preferably human, subject to whom the probiotic composition is administered.

“Urogenital” refers to the urinary and genital systems and organs. The two systems exist in close proximity, and as such, it is helpful to consider the invention as it relates to both. The term “urogenital” may be used interchangeably with “genitourinary” or “urinary and genital”. The urinary system may also be referred to as the “renal system” or “urinary tract”. The genital system may also be referred to as the “reproductive system”.

“UTI” is used interchangeably herein to refer to “urinary tract infection”.

“Recurrent” UTI is repeated UTI, which may be due to relapse or reinfection, and may be defined as 3 or more UTIs in the last 12 months, or 2 or more episodes of confirmed UTI in the last 6 months.

“Relapse” is a recurrent UTI with the same strain of micro-organism. Relapse is the likely cause if infection recurs within a short period after treatment (for example within 2 weeks).

“Reinfection” is a recurrent UTI with a different strain or species of micro-organism. Reinfection is the likely cause if UTI recurs more than 2 weeks after treatment.

The following embodiments are embodiments according to each aspect of the present invention, and any embodiment may be combined with any other embodiment unless explicitly stated otherwise.

In order for probiotics to work effectively and to their optimum, they need to survive the conditions of the upper gastrointestinal (GI) tract without triggering digestion. If digestion is triggered, the stomach acids can weaken or destroy probiotic bacteria. This is a particular problem for probiotics formulated as yoghurt-type drinks, which are known to trigger production of stomach acid, pepsin and other digestive compounds.

In contrast, administration of a liquid, non-dairy probiotic composition in accordance with the methods of the invention results in the bacteria in the composition surviving the conditions of the GI tract and establishing in both the mucosal and luminal compartments of the gut.

Without wishing to be bound by theory, it is hypothesised that because the probiotic bacteria are able to establish in the luminal compartment and especially in the mucosa, the probiotic effect is able to be persistent and maintained. This is in contrast with the effect seen with many probiotics, where the inability to influence the gastrointestinal microbiota as a whole, and the mucosal microbiota in particular, means any effect is restricted to a transient effect on the luminal compartment. This is because any effect of such alternative probiotics will only occur during the transit of the probiotic bacteria through the digestive tract. Moreover, the probiotic compositions provided herein will also result in reduced urogenital tract infection and or inflammation, for example through modulation of the urogenital tract microbiota. Treatment of urogenital infection and/or urogenital inflammation will be particularly effective as a result, for example, of the improved establishment of the probiotic bacteria in the gut allowing the subject to benefit from an interaction of the gut, urinary tract and genital tract microbiota leading to improved urogenital health. In addition, the effects of a PAC and/or mannose in the composition will also prevent and/or treat urogenital infection and/or inflammation.

### Probiotic Compositions

Thus, in a first aspect, the invention provides a liquid, non-dairy probiotic composition comprising a population of viable lactic acid bacteria, and mannose.

In a second aspect, the invention provides a liquid, non-dairy probiotic composition comprising a population of viable lactic acid bacteria, and a proanthocyanidin (PAC).

In a third aspect, the invention provides a liquid, non-dairy probiotic composition comprising a population of viable lactic acid bacteria, a proanthocyanidin (PAC), and mannose.

In certain embodiments of the first, second and third aspects, the population of viable lactic acid bacteria comprises one or more Lactobacillus species. In certain embodiments, the population of viable lactic acid bacteria comprises one or more of Lactobacillus rhamnosus, Lactobacillus acidophilus, and Lactobacillus plantarum bacteria. For example, in certain embodiments, the population of viable lactic acid bacteria comprises Lactobacillus rhamnosus bacteria. In certain embodiments, the population of viable lactic acid bacteria comprises Lactobacillus acidophilus bacteria. In certain embodiments, the population of viable lactic acid bacteria comprises Lactobacillus plantarum bacteria.

In preferred embodiments, the population of viable lactic acid bacteria comprises two or more of Lactobacillus rhamnosus, Lactobacillus acidophilus, and Lactobacillus plantarum bacteria. In more preferred embodiments, the population of viable lactic acid bacteria comprises each of Lactobacillus rhamnosus, Lactobacillus acidophilus, and Lactobacillus plantarum bacteria.

In certain embodiments, the population of viable lactic acid bacteria comprises one or more Enterococcus species. In preferred embodiments, the population of viable lactic acid bacteria comprises Enterococcus faecium bacteria.

In more preferred embodiments, the population of viable lactic acid bacteria comprises each of Lactobacillus rhamnosus, Lactobacillus acidophilus, Lactobacillus plantarum, and Enterococcus faecium bacteria.

In preferred embodiments, the lactic acid bacteria are metabolically active.

In certain aspects and embodiments, the probiotic composition comprises mannose.

In certain embodiments, the concentration of mannose in the probiotic composition is about 2.8 mg/mL to about 280 mg/mL. In certain such embodiments, the concentration of mannose is about 5.6 mg/mL to about 140 mg/mL. In certain such embodiments, the concentration of mannose is about 11 mg/mL to about 70 mg/mL. In preferred embodiments, the concentration of mannose in the probiotic composition is about 28 mg/mL.

In certain embodiments, the probiotic composition comprises a disaccharide, oligosaccharide or polysaccharide comprising at least one mannose residue.

In certain embodiments, the probiotic composition comprises a disaccharide, oligosaccharide or polysaccharide comprising at least one mannose residue, wherein the mannose concentration thereof is about 2.8 mg/mL to about 280 mg/mL. In certain such embodiments, the mannose concentration is about 5.6 mg/mL to about 140 mg/mL. In certain such embodiments, the mannose concentration is about 11 mg/mL to about 70 mg/mL. In preferred such embodiments, the mannose concentration is about 28 mg/mL. The “mannose concentration thereof” refers to the total concentration of mannose in the probiotic composition.

In certain aspects and embodiments, the probiotic composition comprises a proanthocyanidin (PAC).

In certain embodiments, the PAC concentration in the probiotic composition is about 0.04 mg/mL to about 5 mg/mL. In certain such embodiments, the PAC concentration is about 0.1 mg/mL to about 2.5 mg/mL. In certain such embodiments, the PAC concentration is about 0.2 mg/mL to about 1.3 mg/mL. In certain such embodiments, the PAC concentration is about 0.4 mg/mL to about 0.6 mg/mL. In preferred embodiments, the PAC concentration in the probiotic composition is about 0.5 mg/mL.

In certain embodiments, the PAC is obtainable from cranberries.

In certain embodiments, the probiotic composition comprises a cranberry product. In preferred such embodiments, the cranberry product comprises the PAC. In certain embodiments, the cranberry product is a cranberry extract. A cranberry extract may comprise a cranberry juice (i.e. a soluble fraction), a cranberry pomace (i.e. an insoluble fraction), or both a cranberry juice and a cranberry pomace. In preferred embodiments, the cranberry extract comprises or consists of cranberry juice. In certain embodiments, the cranberry extract comprises cranberry juice and one or more excipients.

In preferred embodiments, the cranberry product (e.g. a cranberry extract) is present in the composition in an amount sufficient to provide the probiotic composition with a PAC concentration of about 0.04 mg/mL to about 5 mg/mL, preferably about 0.1 mg/mL to about 2.5 mg/mL, more preferably about 0.2 mg/mL to about 1.3 mg/mL, even more preferably about 0.4 mg/mL to about 0.6 mg/mL, most preferably about 0.5 mg/mL.

In certain embodiments, the cranberry product (e.g. a cranberry extract) is present in the probiotic composition at a concentration of about 0.1 mg/mL to about 14 mg/mL. In certain such embodiments, the cranberry product is present at a concentration of about 0.3 mg/ml to about 7 mg/mL. In certain such embodiments, the cranberry product is present at a concentration of about 0.6 mg/mL to about 3.5 mg/mL. In certain such embodiments, the cranberry product is present at a concentration of about 1 mg/ml to about 1.8 mg/mL. In preferred embodiments, the cranberry product is present in the probiotic composition at a concentration of about 1.4 mg/mL.

In certain embodiments, the population of viable lactic acid bacteria comprises one or more Lactobacillus species. In certain embodiments, the population of viable lactic acid bacteria comprises one or more of Lactobacillus rhamnosus, Lactobacillus acidophilus, and Lactobacillus plantarum bacteria. For example, in certain embodiments, the population of viable lactic acid bacteria comprises Lactobacillus rhamnosus bacteria. In certain embodiments, the population of viable lactic acid bacteria comprises Lactobacillus acidophilus bacteria. In certain embodiments, the population of viable lactic acid bacteria comprises Lactobacillus plantarum bacteria.

In preferred embodiments, the population of viable lactic acid bacteria comprises two or more of Lactobacillus rhamnosus, Lactobacillus acidophilus, and Lactobacillus plantarum bacteria. In more preferred embodiments, the population of viable lactic acid bacteria comprises each of Lactobacillus rhamnosus, Lactobacillus acidophilus, and Lactobacillus plantarum bacteria.

In certain embodiments, the population of viable lactic acid bacteria comprises one or more Enterococcus species. In preferred embodiments, the population of viable lactic acid bacteria comprises Enterococcus faecium bacteria.

In preferred embodiments, the lactic acid bacteria are metabolically active.

In a fourth aspect, the invention provides a liquid, non-dairy probiotic composition comprising a viable population of lactic acid bacteria for use in a method of treatment or prevention of urogenital infection and/or urogenital inflammation.

In a fifth aspect, the invention provides a liquid, non-dairy probiotic composition according to the first, second or third aspects of the invention, which is for use in a method of treatment or prevention of urogenital infection and/or urogenital inflammation.

In a sixth aspect, the invention provides a method of treating or preventing urogenital infection and/or urogenital inflammation in a subject in need thereof, the method comprising administering a liquid, non-dairy probiotic composition comprising a viable population of lactic acid bacteria to the subject.

In a seventh aspect, the invention provides a method of treating or preventing urogenital infection and/or urogenital inflammation in a subject in need thereof, the method comprising administering to the subject a probiotic composition as defined in respect of any of the first, second or third aspects of the invention.

In preferred embodiments of the fourth, fifth, sixth and seventh aspects of the invention, the methods and probiotic compositions treat or prevent urogenital infection. In certain embodiments, the methods and probiotic compositions treat or prevent bacterial or fungal urogenital infection. In certain such embodiments, the methods and probiotic compositions treat or prevent a bacterial or fungal infection of the urinary tract. Preferably, the methods and probiotic compositions treat or prevent a bacterial infection of the urinary tract (e.g. infection of the kidneys, ureters, bladder, and/or urethra). In most preferred embodiments, the methods and probiotic compositions treat or prevent a UTI.

In certain embodiments, the UTI may be recurrent. In certain such embodiments, the UTI may be a relapse. In other such embodiments, the UTI may be a reinfection.

It is well understood that urogenital infection (e.g. UTI) is commonly associated with urogenital inflammation. In certain embodiments, the methods and probiotic compositions treat or prevent urogenital inflammation (e.g. inflammation of the urinary tract) that is comorbid with urogenital infection. In certain embodiments, the subject has received an initial course of treatment, but the infection and inflammation persist. In other embodiments, the urogenital infection has subsided (e.g. following an initial course of treatment), while the inflammation persists.

Urogenital inflammation may also have unknown or unclear causes. In certain embodiments, the subject suffers from urogenital inflammation without a diagnosis of a urogenital infection (e.g. UTI). In some such embodiments, the subject has a suspected UTI. In other such embodiments, the subject is not suspected to have a UTI (e.g. where the subject produces a negative urine culture when testing for microbial infection).

In certain embodiments, the methods and probiotic compositions treat or prevent a sexually-transmitted infection.

In certain embodiments, the methods and probiotic compositions treat or prevent urethritis, cystitis, ureteritis, pyelitis, pyelonephritis, asymptomatic bacteriuria, symptomatic bacteriuria, vaginitis, cervicitis, bacterial vaginosis, candiduria, candidiasis, aerobic vaginitis, epididymitis, orchitis, epididymo-orchitis, prostatitis, interstitial cystitis, nonbacterial cystitis, interstitial nephritis, interstitial urethritis, urethral syndrome or nosocomial UTI. In certain embodiments, the methods and probiotic compositions treat or prevent chlamydia infection, gonorrhoea infection, syphilis infection, mycoplasma infection, ureaplasma infection, urinary schistosomiasis, or trichomoniasis.

Unless specified otherwise herein, all embodiments of the fourth and fifth aspects are equally applicable to the sixth and seventh aspects of the invention and vice-versa.

In certain embodiments, the methods and probiotic compositions reduce the number of UTI episodes experienced by a subject. In certain embodiments, the methods and probiotic compositions reduce the duration of UTI episodes experienced by a subject. In certain embodiments, the methods and probiotic compositions reduce the pain burden of UTI (severity of pain or burning felt when passing urine). In certain embodiments, the methods and probiotic compositions reduce the frequency burden of UTI (frequency of urination of small volumes of urine). In certain embodiments, the methods and probiotic compositions reduce the urgency burden of UTI (a strong and uncontrollable urge to pass urine). In such embodiments, the reduction in number, duration and/or symptoms of UTIs may be determined over a six-month period. For example, the reduction in number, duration and/or symptoms of UTI may be determined over a six-month period in which a subject is administered a 70 mL dose of the probiotic composition per day, relative to a six-month period in which the subject is not administered the probiotic composition. In certain embodiments, the methods and probiotic compositions reduce the probability of a recurrent UTI. For example, the methods and probiotic compositions reduce the probability of a recurrent UTI in a six-month period compared to a control population not administered the probiotic composition.

### Kits

In an eighth aspect, the invention provides a kit comprising:
(i) a first liquid, non-dairy composition comprising a population of viable lactic acid bacteria;
(ii) a second composition comprising mannose.

In certain embodiments, the population of viable lactic acid bacteria comprises one or more Lactobacillus species.

In certain embodiments, the population of viable lactic acid bacteria comprises one or more of Lactobacillus rhamnosus, Lactobacillus acidophilus, and Lactobacillus plantarum bacteria.

In certain embodiments, the population of viable lactic acid bacteria comprises each of Lactobacillus rhamnosus, Lactobacillus acidophilus, and Lactobacillus plantarum bacteria.

In certain embodiments, the population of viable lactic acid bacteria comprises one or more Enterococcus species. In certain embodiments, the population of viable lactic acid bacteria comprises Enterococcus faecium bacteria.

In certain embodiments, the population of viable lactic acid bacteria comprises each of Lactobacillus rhamnosus, Lactobacillus acidophilus, Lactobacillus plantarum and Enterococcus faecium bacteria.

In certain embodiments, the lactic acid bacteria are metabolically active.

In certain embodiments, the mannose concentration is about 2.8 mg/mL to about 280 mg/mL, optionally about 5.6 mg/mL to about 140 mg/mL, optionally about 11 mg/mL to about 70 mg/mL, preferably about 28 mg/mL.

In certain embodiments, the first and second compositions are formulated in combination (i.e. co-formulated). In alternative embodiments, the first and second compositions are formulated separately.

In certain embodiments, the kit comprises a third composition comprising a proanthocyanidin (PAC).

In certain such embodiments, any two of the first, second and third compositions are formulated in combination (i.e. co-formulated). In certain embodiments, the first composition is formulated in combination with the second composition. In other embodiments, the first composition is formulated in combination with the third composition. In other embodiments, the second composition is formulated in combination with the third composition.

In certain embodiments, the first, second and third compositions are co-formulated in a single composition. In certain embodiments, the first, second and third compositions are each formulated separately.

In certain embodiments, the PAC is obtainable from cranberries. In certain embodiments, the third composition comprises a cranberry product comprising said PAC, preferably wherein the cranberry product is a cranberry extract.

In certain embodiments, the PAC is present at a concentration of about 0.04 mg/ml to about 5 mg/mL, optionally about 0.1 mg/mL to about 2.5 mg/mL, optionally about 0.2 mg/mL to about 1.3 mg/mL, optionally about 0.4 mg/mL to about 0.6 mg/mL, preferably about 0.5 mg/mL.

In certain embodiments, the PAC is comprised in a cranberry extract, wherein the cranberry extract is present at a concentration of about 0.1 mg/mL to about 14 mg/mL, optionally about 0.3 mg/mL to about 7 mg/mL, optionally about 0.6 mg/ml to about 3.5 mg/mL, optionally about 1 mg/mL to about 1.8 mg/mL, preferably about 1.4 mg/mL.

In certain embodiments the kit of the eighth aspect can be used in a method of treatment or prevention according to any of the fourth, fifth, sixth or seventh aspects of the invention.

### Composition of the Probiotic Composition

The probiotic composition used in accordance with the invention is a liquid product (i.e. not freeze-dried) which is non-dairy. Preferably the probiotic composition is water-based.

The probiotic composition contains a population of lactic acid bacteria. The lactic acid bacteria are viable probiotic bacteria and are therefore “alive” and metabolically active. In this way the bacteria are ready to function immediately after the composition is swallowed.

In certain embodiments, the probiotic bacteria are of the genus Lactobacillus or Enterococcus. In certain preferred embodiments, the population of lactic acid bacteria comprises one or more of Enterococcus faecium, Lactobacillus plantarum, Lactobacillus acidophilus and Lactobacillus rhamnosus.

Reference to Lactobacillus rhamnosus is intended to include any bacterial strain originally classified as Lactobacillus casei but now re-classified as Lactobacillus rhamnosus.

In certain preferred embodiments, the population of lactic acid bacteria comprises at least two or at least three of Enterococcus faecium, Lactobacillus plantarum, Lactobacillus rhamnosus and Lactobacillus acidophilus. In certain preferred embodiments, the population of lactic acid bacteria comprises Lactobacillus plantarum, Lactobacillus rhamnosus and Lactobacillus acidophilus.

In certain preferred embodiments, the population of lactic acid bacteria comprises each of Enterococcus faecium, Lactobacillus plantarum, Lactobacillus rhamnosus and Lactobacillus acidophilus.

In certain embodiments, the total population of metabolically active bacteria in the probiotic composition may be in the range of from 1.0×10⁶ to 1.0×10¹⁰ viable cells per millilitre, preferably from 1.0×10⁶ to 1.0×10⁹ viable cells per millilitre, preferably in the range of from 1.0×10⁷ to 1.0×10⁹ viable cells per millilitre. Each individual strain of metabolically active bacteria present in the probiotic composition independently may be present in the range of from 1.0×10⁵ to 1.0×10⁹ viable cells per millilitre, more preferably in the range of from 1.0×10⁷ to 1.0×10⁹ viable cells per millilitre.

In the case of probiotic compositions comprising a combination of Lactobacillus plantarum and Lactobacillus rhamnosus or a combination of Enterococcus faecium, Lactobacillus plantarum, and Lactobacillus rhamnosus, it is preferred that at least one and preferably each of these strains is present in the range of from 1.0×10⁶ to 1.0×10¹⁰ viable cells per millilitre, preferably in the range of from 1.0×10⁷ to 1.0×10⁹ viable cells per millilitre.

In certain embodiments, the population of L. acidophilus, if included, may be lower than 1.0×10⁵ viable cells per millilitre, preferably in the range of from 1.0×10² to 1.0×10⁵ viable cells per millilitre, optionally 1.0×10² to 1.0×10⁴ viable cells per millilitre.

In an exemplary embodiment, the probiotic composition may comprise a combination of Enterococcus faecium, Lactobacillus plantarum, and Lactobacillus rhamnosus, wherein the bacterial count for each of these bacterial strain is in the range of from 1.0×10⁵ to 1.0×10⁹ viable cells per millilitre, more preferably in the range of from 1.0×10⁷ to 1.0×10⁹ viable cells per millilitre. The population of L. acidophilus, if included, may be lower than 1.0×10⁵ viable cells per millilitre, preferably in the range of from 1.0×10² to 1.0×10⁵ viable cells per millilitre, optionally 1.0×10² to 1.0×10⁴ viable cells per millilitre.

In certain embodiments, the probiotic composition comprises a plant product as defined herein. In certain embodiments, the plant product is a plant extract, such as the juice extracted from the fruit of the plant. In other embodiments, the plant product consists of whole or part of a plant, such as the fruit of the plant. In certain embodiments, the probiotic composition may be prepared using a dried plant product (e.g. dried plant extract).

In certain embodiments, the plant product is an edible berry of the Vaccinium genus. In preferred embodiments, the plant product is a cranberry of the Vaccinium genus. In certain embodiments, the cranberry is of the Vaccinium oxycoccos species. In other embodiments, the cranberry is of the Vaccinium microcarpum species. In other embodiments, the cranberry is of the Vaccinium erythrocarpum species. In preferred embodiments, the cranberry is of the Vaccinium macrocarpon species. In certain embodiments, the plant product comprises cranberries of two or more species selected from: Vaccinium oxycoccos, Vaccinium erythrocarpum, Vaccinium microcarpum and Vaccinium macrocarpon.

In most preferred embodiments, the plant product (e.g. a cranberry extract) comprises a PAC. In preferred embodiments, the plant product (e.g. cranberry extract) is present in the composition in an amount sufficient to provide the probiotic composition with a PAC concentration of about 0.04 mg/mL to about 5 mg/mL, preferably about 0.1 mg/mL to about 2.5 mg/mL, more preferably about 0.2 mg/mL to about 1.3 mg/mL, even more preferably about 0.4 mg/mL to about 0.6 mg/mL, most preferably about 0.5 mg/mL.

In certain most preferred embodiments, the probiotic composition is Symprove™ to which Vaccinium macrocarpon (cranberry) extract containing a PAC, and/or mannose has been added.

In certain embodiments, the concentration of mannose in the probiotic composition is about 2.8 mg/mL to about 280 mg/mL. In certain such embodiments, the concentration of mannose is about 5.6 mg/mL to about 140 mg/mL. In certain such embodiments, the concentration of mannose is about 11 mg/mL to about 70 mg/mL. In preferred embodiments, the concentration of mannose in the probiotic composition is about 28 mg/mL.

In certain preferred embodiments, the probiotic composition typically contains a mixture of polysaccharides, oligosaccharides, disaccharides and monosaccharides.

In certain embodiments, the probiotic composition may be characterised by the ratio of total carbohydrate content to reducing sugar content of the composition, reflecting the complex mix of polysaccharides, oligosaccharides, disaccharides and monosaccharides present.

In certain embodiments, when the probiotic composition does not contain mannose, the ratio of total carbohydrate content to reducing sugar content of the composition is in the range of from 8:1 to 2:1, more typically in the range of from 5:1 to 2.5:1, or in the range of from 4:1 to 3:1.

In further exemplary embodiments of the probiotic composition, when the probiotic composition does not contain mannose, the total carbohydrate (polysaccharides, oligosaccharides, disaccharides and monosaccharides) content of the composition may be in the range of from 20 mg/mL to 40 mg/mL, or in the range of from 20 mg/mL to 30 mg/mL and the total reducing sugar content may be in the range of from 5 mg/mL to 20 mg/mL, or in the range of from 5 mg/mL to 10 mg/mL.

In certain embodiments, when the probiotic composition comprises mannose, the ratio of total carbohydrate content to reducing sugar content of the composition is in the range of from 3:1 to 1:1, more typically in the range of from 2:1 to 1:1, or in the range of from 1.5:1 to 1:1.

In further exemplary embodiments of the probiotic composition, when the probiotic composition comprises mannose, the total carbohydrate (polysaccharides, oligosaccharides, disaccharides and monosaccharides) content of the composition may be in the range of from 20 mg/mL to 320 mg/mL, from 25 mg/mL to 260 mg/mL, from 30 mg/mL to 200 mg/mL, from 35 mg/mL to 140 mg/mL, or from 40 mg/mL to 80 mg/mL, and the total reducing sugar content may be in the range of from 20 mg/mL to 320 mg/mL, 22 mg/mL to 270 mg/mL, 24 mg/mL to 220 mg/mL, 26 mg/mL to 170 mg/mL, 28 mg/mL to 120 mg/mL, or from 30 mg/mL to 70 mg/mL.

The probiotic composition preferably also comprises protein and peptide components. Typically the total amount of protein and peptides present in the probiotic composition is in the range of from 0.01 mg/mL to 2 mg/mL, preferably 0.05 mg/mL to 2 mg/mL. Preferably the total amount of high molecular weight peptides (molecular weight greater than 5000 Daltons) is in the range of from 10 μg/mL to 300 μg/mL, preferably of from 50 μg/mL to 200 μg/mL. In a specific embodiment, the concentration of protein and peptides may be about 1 mg/mL to about 2 mg/mL and the concentration of high molecular weight peptides may be about 250 μg/mL.

Means of measuring the concentrations of these nutritional components are provided in WO 2006/035218, which is incorporated herein by reference.

The probiotic composition may contain further components such as, for example, cellulose, starch, β-glucans, pentosans, polyphenols other than PACs, ribonucleic acids, lipids, phosphates, flavonoids other than PACs, amino acids, vitamins (B₁, B₂, C and E), silicates and trace elements.

The probiotic composition may comprise an extract of germinated barley containing the desired probiotic bacterial strains.

An exemplary embodiment of the probiotic composition comprises extract of germinated barley and a combination of Enterococcus faecium, Lactobacillus plantarum, Lactobacillus rhamnosus, wherein the bacterial count for each of these bacterial strain is in the range of from 1.0×10⁵ to 1.0×10¹⁰ viable cells per millilitre, more preferably in the range of from 1.0×10⁷ to 1.0×10⁹ viable cells per millilitre, and further contains Lactobacillus acidophilus at a concentration of lower than 1.0×10⁵ viable cells per millilitre, preferably in the range of from 1.0×10² to 1.0×10⁵ viable cells per millilitre, optionally 1.0×10² to 1.0×10⁴ viable cells per millilitre.

Additional components may be added to the probiotic composition, such as flavourings and/or colourings, to improve palatability.

The pH of the composition can be conveniently controlled by the addition of a suitable buffer or combination of buffering agents. Preferred buffers include, for example, tri-sodium citrate or phosphate buffers. The pH of the liquid-based composition described herein is typically maintained in the range of from 3.8 to 4.5, and in particular at about pH 4.0.

The probiotic composition may be stored at any temperature from 4° C. up to ambient temperature (about 25° C.). The Symprove™ product described herein has been shown to remain stable (in terms of bacterial count) for a period of at least 6 months when stored at about 4° C., and for at least 4 months when stored at 25° C.

The probiotic composition may additionally comprise an anti-fungal agent, such as, for example, sterilised potassium sorbate and/or an anti-oxidant, such as vitamin C.

In a preferred embodiment, the growth substrate may contain particulate matter, for example particles not exceeding 1 mm in diameter.

In most preferred embodiments of the first, second and third aspects, the probiotic composition is the product denoted Symprove™ further comprising a PAC and/or mannose. The original Symprove™ product does not contain mannose. Symprove™ contains viable cells of Enterococcus faecium, Lactobacillus plantarum, Lactobacillus rhamnosus and Lactobacillus acidophilus, which may be prepared according to the examples provided in WO 2006/035218 (incorporated by reference and discussed elsewhere herein). The strain of Lactobacillus rhamnosus in Symprove™ was originally characterised as Lactobacillus casei but has now been re-classified as the closely related Lactobacillus rhamnosus.

### Administration

In certain preferred embodiments, the probiotic composition is administered orally.

In certain embodiments, the probiotic composition is administered at least once a week. In certain preferred embodiments, the probiotic composition is administered at least once a day, preferably once a day.

In certain preferred embodiments, the probiotic composition is administered in the range of from 0.5 mg/kg of the subject to 5 mg/kg of the subject. In a preferred embodiment, the probiotic composition is administered at a dose of 1 mg/kg of the subject.

In certain preferred embodiments, the probiotic composition is administered to the subject at dose of 40-100 mL/day, preferably 70 mL/day.

In certain embodiments, the probiotic composition is administered to the subject in a dose providing about 7.2 to about 180 mg PAC/day, about 14.4 to about 90 mg PAC/day, about 28.8 to about 45 mg PAC/day, about 30 to about 42 mg PAC/day, or about 36 mg PAC/day.

In certain preferred embodiments, the probiotic composition is administered to the subject in a dose providing at least 36 mg PAC/day.

In certain preferred embodiments, the probiotic composition is administered for at least 1 week, preferably at least 2 weeks, preferably at least 3 weeks, preferably at least 4 weeks. In certain preferred embodiments, the composition is administered for at least 5, at least 6, at least 7, at least 8, at least 9, at least 10, at least 11 or at least 12 weeks. In certain preferred embodiments, the composition is administered for at least 1, at least 2, at least 3, at least 4, at least 5 or at least 6 months. Preferably the probiotic composition is administered once daily for the indicated time period.

In most preferred embodiments, the probiotic composition is administered at a dose of 1 mg/kg of the subject or 70 mL per day, at least once a day for at least 3 weeks, preferably at least 1 month.

### Manufacture of the Probiotic Composition

A probiotic composition of the invention may be prepared by addition of a PAC and/or mannose to a base composition. Exemplary methods for production of a base composition are provided in WO 2006/035218, the contents of which are incorporated herein by reference. For example, a base composition can be prepared by growing one or more probiotic bacterial strains in a liquid growth substrate, such as for example an extract of germinated barley. The growth substrate may be itself prepared starting from seed or malting sample barley using the manufacturing process described in WO 2006/035218, the contents of which are incorporated herein by reference.

Once the base composition is prepared, a PAC may be added, such as by addition of a PAC-containing cranberry extract to the substrate. Mannose may be added to the liquid growth substrate in addition to or alternatively to the PAC. Suitable concentrations of the PAC (e.g. in PAC-containing cranberry extract) and mannose are described herein.

An alternative method of producing the probiotic composition which does not require an active growing step simply involves preparing a base composition by inoculating a growth substrate (e.g. the extract of germinated barley prepared as described in WO2006/035218) with starter culture(s) of probiotic bacteria. Starter cultures for the method include, for example, freeze-dried bacteria or liquid cultures. The growth substrate may be inoculated with more than one bacterial species. Preferably, the concentration of the viable cells in the starter culture is in excess of 10⁶ viable cells per millilitre. This method is also described in WO 2006/035218.

A further alternative method of producing the probiotic composition is to prepare a base composition by compiling a nutrient substrate meeting the nutrient requirements of the probiotic composition described herein and also in WO 2006/035218, and inoculating said substrate with the probiotic bacteria.

After inoculation with the probiotic bacteria, a PAC may be added to the base composition, such as by addition of a PAC-containing cranberry extract. Mannose may additionally or alternatively be added to the composition. Suitable concentrations of the PAC, cranberry extract and mannose are described herein.

A PAC (or a PAC-containing cranberry extract) and mannose may each independently be in the form of a liquid or a powder to be dissolved in the composition.

The invention will be further understood with reference to the following non-limiting experimental examples:

### EXAMPLES

### Method

A two-arm randomized double-blind placebo-controlled superiority trial was conducted in subjects with UTI.

The study included 139 participants. The participants were women in the community between the ages of 20 and 75 years who had experienced two or more episodes of UTI in the six months prior to commencement of the study (i.e. recurrent UTI).

Participants were randomised into a placebo/control group, or an intervention/active group. At the end of the study, the placebo/control group contained 74 participants and the intervention/active group contained 65 participants. The intervention/active group received the probiotic composition described herein.

Specifically, participants in the intervention/active group were administered 70 mL per day (for six months) of a probiotic composition comprising live Enterococcus faecium, Lactobacillus plantarum, Lactobacillus rhamnosus and Lactobacillus acidophilus. The probiotic composition further comprised approximately 2 g mannose and approximately 98 mg cranberry extract. The cranberry extract was in the form of powdered cranberry juice obtained from cranberry fruit (Vaccinium macrocarpon), and provided approximately 36 mg total PAC per 70 mL dose of the probiotic composition.

The placebo/control group beverage was a liquid which is comparable to the liquid consumed by the intervention/active group (e.g. having the same appearance, taste, and consistency), but notably lacking live bacteria, cranberry extract and mannose.

### Data Collection

Self-reported UTI symptom data was collected via an Acute Cystitis Symptom Score (ACSS) questionnaire completed on each day that a particular participant experienced UTI symptoms.

### Results

By the end of the study, the control group reported a total of 60 UTIs whereas the intervention group reported a total of 39 UTIs. The average duration of each UTI event in the control group was 4 days whereas the average duration in the intervention group was 2.3 days. The total duration of UTIs in the control group was 185 days whereas in the intervention group, it was 100 days.

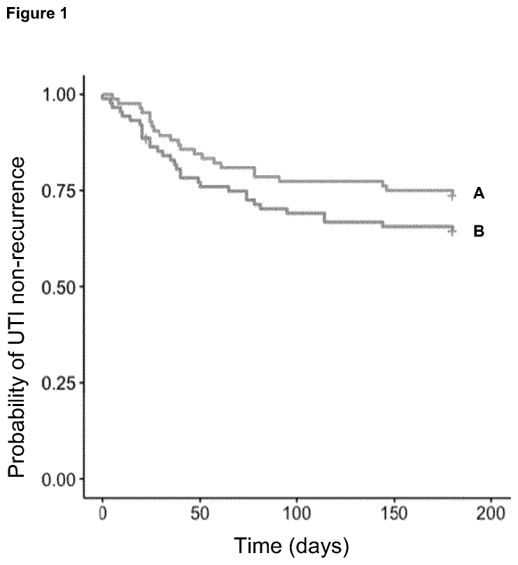

By the end of the six-month trial, 92% of participants in the intervention group noticed a reduction in the number of UTI episodes compared to the number of episodes in the six months prior to commencement of the trial. In contrast, only 85% of participants in the placebo group noticed a reduction in the number of UTI episodes compared to the number of episodes in the six months prior to commencement of the trial. As shown in FIG. 1, the probability of non-recurrence of UTI was higher in the intervention group (“A”) than the control group (“B”) over the six-month period.

Pain burden (feeling pain or burning when passing urine) was reported for each day of a UTI event and scored on the following scale: 0=No, 1=Mild, 2=Moderate, and 3=Severe. The total pain burden reported in the control group over the six-month period was 211 whereas the total pain burden in the intervention group was 106.

Frequency burden (frequent urination of small volumes of urine) was reported for each day of a UTI event and scored on the following scale: 0=No (4 or less times per day), 1=Mild (5-6 times per day), 2=Moderate (7-8 times per day), and 3=Severe (9-10 times per day). The total frequency burden in the control group over the six-month period was 283 whereas the total frequency burden in the intervention group was 153.

Urgency burden (a strong and uncontrollable urge to pass urine) was reported for each day of a UTI event and scored on the following scale: 0=No, 1=Mild, 2=Moderate, and 3=Severe. The total urgency burden in the control group over the six-month period was 263 whereas the total frequency burden in the intervention group was 137.

TABLE 1
results scaled to account for the difference in the number of participants between
the intervention (n = 65) and control (n = 74) groups.

| | | Average | | | | |
|---|---|---|---|---|---|---|
| | Number | UTI | Total UTI | Pain | Frequency | Urgency |
| | of UTIs | Duration | Duration | burden | burden | Burden |
| Intervention | 44 | 2.6 | 114 | 121 | 174 | 157 |
| Control | 60 | 4.0 | 185 | 211 | 283 | 263 |

The results of the trial show that relative to control, the probiotic composition was more effective overall at reducing both the number and duration of UTI events, as well the severity of UTI symptoms in participants who experienced UTI.

### CONCLUSION

The probiotic compositions described herein are an effective treatment/prophylaxis for UTI and related conditions, improving overall quality of life for individuals with UTI and/or a related condition.

1. A liquid, non-dairy probiotic composition comprising a population of viable lactic acid bacteria and mannose.

2. The composition of claim 1, wherein the population of viable lactic acid bacteria comprises one or more Lactobacillus species.

3. The composition of claim 1 or claim 2, wherein the population of viable lactic acid bacteria comprises one or more of Lactobacillus rhamnosus, Lactobacillus acidophilus, and Lactobacillus plantarum bacteria.

4. The composition of any preceding claim, wherein the population of viable lactic acid bacteria comprises one or more Enterococcus species.

5. The composition of any preceding claim, wherein the population of viable lactic acid bacteria comprises Enterococcus faecium bacteria.

6. The composition of any preceding claim, wherein the population of viable lactic acid bacteria comprises each of Lactobacillus rhamnosus, Lactobacillus acidophilus, Lactobacillus plantarum and Enterococcus faecium bacteria.

7. The composition of any preceding claim, comprising a proanthocyanidin (PAC).

8. The composition of claim 7, wherein the PAC is obtainable from cranberries.

9. The composition of claim 7 or claim 8, comprising a cranberry product comprising said PAC, preferably wherein the cranberry product is a cranberry extract.

10. The composition of any preceding claim comprising a disaccharide, oligosaccharide or polysaccharide comprising at least one mannose residue.

11. The composition of any preceding claim, wherein the lactic acid bacteria are metabolically active.

12. The composition of any preceding claim, wherein the mannose concentration is about 2.8 mg/mL to about 280 mg/mL, optionally about 5.6 mg/mL to about 140 mg/mL, optionally about 11 mg/mL to about 70 mg/mL, preferably about 28 mg/mL.

13. The composition of any one of claims 7-9, wherein the PAC is present at a concentration of about 0.04 mg/mL to about 5 mg/mL, optionally about 0.1 mg/mL to about 2.5 mg/mL, optionally about 0.2 mg/mL to about 1.3 mg/mL, optionally about 0.4 mg/mL to about 0.6 mg/mL, preferably about 0.5 mg/mL.

14. The composition of any one of claim 7-9 or 13, wherein the PAC is comprised in a cranberry extract, wherein the cranberry extract is present at a concentration of about 0.1 mg/mL to about 14 mg/mL, optionally about 0.3 mg/mL to about 7 mg/mL, optionally about 0.6 mg/mL to about 3.5 mg/mL, optionally about 1 mg/mL to about 1.8 mg/mL, preferably about 1.4 mg/mL.

15. A method of treating or preventing urogenital infection and/or urogenital inflammation in a subject in need thereof, the method comprising administering a liquid, non-dairy probiotic composition comprising a viable population of lactic acid bacteria to the subject.

16. The method of claim 15, wherein the method is a method of treating or preventing urogenital infection, wherein said urogenital infection is a urinary tract infection (UTI).

17. The method of claim 16, wherein the method reduces one or more of number of UTI episodes, duration of UTI, pain burden, frequency burden and urgency burden.

18. The method of any one of claims 15-17, wherein the composition is as defined in any one of claims 1-14.

19. A liquid, non-dairy probiotic composition comprising a viable population of lactic acid bacteria for use in a method of treatment of urogenital infection and/or urogenital inflammation.

20. The composition for use according to claim 19, wherein the composition is for use in treating a urogenital infection, wherein said urogenital infection is a urinary tract infection (UTI).

21. The composition for use according to claim 20, wherein the subject exhibits a reduction in one or more of number of UTI episodes, duration of UTI, pain burden, frequency burden and urgency burden.

22. The composition for use according to any one of claims 19-21, wherein the composition is as defined in any one of claims 1-14.

23. Use of a liquid, non-dairy probiotic composition comprising a viable population of lactic acid bacteria in the manufacture of a medicament for treating or preventing urogenital infection and/or urogenital inflammation.

24. The use according to claim 23, wherein the medicament is for treating a urogenital infection, wherein said urogenital infection is a urinary tract infection (UTI).

25. The use according to claim 24, wherein the medicament reduces one or more of number of UTI episodes, duration of UTI, pain burden, frequency burden and urgency burden.

26. The use according to any one of claims 23-25, wherein the composition is as defined in any one of claims 1-14.

27. A kit comprising:
(i) a first liquid, non-dairy composition comprising a population of viable lactic acid bacteria; and
(ii) a second composition comprising mannose.

28. The kit of claim 27, wherein the population of viable lactic acid bacteria comprises one or more Lactobacillus species.

29. The kit of claim 27 or claim 28, wherein the population of viable lactic acid bacteria comprises one or more of Lactobacillus rhamnosus, Lactobacillus acidophilus, and Lactobacillus plantarum bacteria.

30. The kit of any one of claims 27-29, wherein the population of viable lactic acid bacteria comprises one or more Enterococcus species.

31. The kit of any one of claims 27-30, wherein the population of viable lactic acid bacteria comprises Enterococcus faecium bacteria.

32. The kit of any one of claims 27-31, wherein the population of viable lactic acid bacteria comprises each of Lactobacillus rhamnosus, Lactobacillus acidophilus, Lactobacillus plantarum and Enterococcus faecium bacteria.

33. The kit of any one of claims 27-32, wherein the mannose concentration is about 2.8 mg/mL to about 280 mg/mL, optionally about 5.6 mg/mL to about 140 mg/mL, optionally about 11 mg/mL to about 70 mg/mL, preferably about 28 mg/mL.

34. The kit of any one of claims 27-33, wherein the first and second compositions are formulated in combination.

35. The kit of any one of claims 27-33, wherein the first and second compositions are formulated separately.

36. The kit of any one of claims 27-35 comprising a third composition comprising a proanthocyanidin (PAC).

37. The kit of claim 36, wherein the first and third compositions are formulated in combination.

38. The kit of claim 36, wherein the second and third compositions are formulated in combination.

39. The kit of claim 36, wherein the third composition is formulated separately from the first and second compositions.

40. The kit of any one of claims 36-39, wherein the PAC is obtainable from cranberries.

41. The kit of any one of claims 36-40, wherein the third composition comprises a cranberry product comprising said PAC, preferably wherein the cranberry product is a cranberry extract.

42. The kit of any one of claims 36-41, wherein the PAC is present at a concentration of about 0.04 mg/mL to about 5 mg/mL, optionally about 0.1 mg/mL to about 2.5 mg/mL, optionally about 0.2 mg/mL to about 1.3 mg/mL, optionally about 0.4 mg/mL to about 0.6 mg/mL, preferably about 0.5 mg/mL.

43. The kit of any one of claims 36-42, wherein the PAC is comprised in a cranberry extract, wherein the cranberry extract is present at a concentration of about 0.1 mg/mL to about 14 mg/mL, optionally about 0.3 mg/mL to about 7 mg/mL, optionally about 0.6 mg/mL to about 3.5 mg/mL, optionally about 1 mg/mL to about 1.8 mg/mL, preferably about 1.4 mg/mL.

44. The kit of any one of claims 27-43, wherein the lactic acid bacteria are metabolically active.
